(19) United States
(12) Patent Application Publication
(10) Pub. No.: US 2003/0068660 A1
(43) Pub. Date: Apr. 10, 2003

(54) Methods of ameliorating arthritis by modulating JNK signalsome activity

(76) Inventor: Gary S. Firestein, Del Mar, CA (US)

Correspondence Address: GARY CARY WARE & FRIENDENRICH LLP, 4365 EXECUTIVE DRIVE, SUITE 1600, SAN DIEGO, CA, 92121-2189, US

(21) Appl. No.: 10/241,050

(22) Filed: Sep. 10, 2002

Related U.S. Application Data
(60) Provisional application No. 60/323,195, filed on Sep. 12, 2001.

(51) Int. Cl. G01N 33/567

(52) U.S. Cl. 435/7.21

(57) ABSTRACT

Methods for identifying an agent that modulates JNK signalsome mediated signal transduction are provided. As provided are methods of ameliorating an arthritis in a subject by inhibiting JNK signalsome activity in cells of the subject.

### RELATED APPLICATION DATA

This application claims the benefit under 35 U.S.C. 119(e) of U.S. Serial No. 60/323,195, filed Sep. 12, 2001, which is incorporated herein by reference in its entirety.

### GOVERNMENT SUPPORT

This invention was made in part with government support under Grant No. AR47825 awarded by the National Institutes of Health. The United States government may have certain rights in this invention.

### BACKGROUND OF THE INVENTION

1 Field of the Invention

This invention relates generally to the identification of protein complex that regulate a signal transduction pathway in synoviocytes of subjects with arthritis, and, more specifically, to complexes formed by the specific association of a c-Jun N-terminal kinase (JNK) and at least one MAP kinase kinase (MKK), including, for example, a JNK signalsome comprising a complex of JNK, MKK4 and MKK7; to complexes formed by the specific association of MKK4 or MKK7 with a MAP kinase kinase kinase (MAP3K); to methods of identifying agents that modulate JNK mediated signal transduction in synoviocytes; and to methods of ameliorating a disorder associated with abnormal JNK signalsome mediated signal transduction, including, for example, an arthritis such as rheumatoid arthritis or osteoarthritis.

2. Background Information

Rheumatoid arthritis (RA) is the most common inflammatory arthritis and is characterized by synovial inflammation and hyperplasia. RA is a systemic, chronic, inflammatory disease that generally affects joints of the fingers, toes, elbows, knees, ankles and spine (see Pathology, 3d edition (ed. Rubin and Farber; Lippincott-Raven 1998); pages 1396-1400). RA is more common in women than men, and onset can occur at any age, though it generally begins in the third or fourth decade, with the incidence increasing with age.

A hallmark of chronic RA is expansion of the synovial intimal lining and joint destruction due to increased numbers of both macrophage-like and fibroblast-like synoviocytes. Growth factors, cytokines, adhesion molecules and oncogenes such as c-Myc, c-Fos and c-Jun are expressed by fibroblast-like synoviocytes in the synovium under the influence of inflammatory cytokines. The intimal lining also produces prodigious quantities of proteases that degrade cartilage and bone. Of the many classes of enzymes are involved in this process, the matrix metalloproteinases (MMP) are probably the most important. In situ hybridization studies show that the primary location of collagenase gene expression is the synovial intimal lining, especially in fibroblast-like cells. Collagenase protein has also been detected in the same region using immunohistochemistry. Increased MMP gene expression is a feature of early RA and occurs during the first few weeks or months of disease.

Treatments for rheumatoid arthritis generally are directed to alleviating the symptoms of the disease. Several different classes of drugs utilized to treat patients with the various types of rheumatoid arthritis, including analgesics to control pain, corticosteroids, uric acid-lowering drugs, immunosuppressive drugs, nonsteroidal anti-inflammatory drugs (NSAIDs), and disease-modifying anti-rheumatic drugs (DMARDs). Corticosteroids, for example, act generally to reduce inflammatory responses, and often are prescribed for arthritis patients. However, long term treatment with corticosteroids can produce undesirable side effects. NSAIDs, including, for example, aspirin, ibuprofen, and naproxen, are the most commonly used drugs and are generally well tolerated by patients. NSAIDs decrease the inflammatory response of the body to disease or injury. However, they have little or no effect on the underlying disease and, therefore, cannot prevent progression of joint destruction or organ damage. The effects of NSAIDs are relatively rapid, occurring over a period of a few hours. However, the benefits of NSAIDs rapidly fade when treatment is stopped. In addition, NSAIDs cause a number of side effects including, for example, gastrointestinal tract irritation, skin reactions and rashes, increases in blood coagulation time, reversible hepatocellular toxicity, and impaired renal function.

DMARDs, including, for example, methotrexate and TNF inhibitors, often are used in patients with more advanced disease, and may have some effect on altering the progression of rheumatoid arthritis. However, in contrast to NSAIDs, DMARDs are slower acting, taking weeks or months for benefits of the drug to occur, and they have a higher incidence of toxicity. Because of the toxicity, patients receiving DMARD therapy must be carefully and frequently evaluated. All of the DMARDs have significant side effects, including, for example, retinal toxicity with the antimalarial drugs, dermatitis or other skin rashes, nausea, diarrhea, and various types of anemia.

Ideally, rheumatoid arthritis would be treated by identifying the molecular basis for the disease and developing drugs that specifically target the intracellular pathways involved in the etiology of the disease. In this respect, a signal transduction enzyme, JNK, has been shown to be involved in the regulation of gene expression that leads to inflammation and joint destruction in arthritis. As such, the JNK enzyme provides a potential target for drugs that can inhibit JNK activity and, therefore, the signal transduction pathways responsible for producing the tissue damage associated with arthritis. Unfortunately, there a multiple different isoforms of JNK expressed in cells, and many of the isoforms mediate important signal transduction pathways required to maintain the viability of cells and organisms. As such, while an effort can be made to identify drugs that inhibit only the JNK isoform that is involved in the tissue damage associate with rheumatoid arthritis, such selective agents are likely to be difficult to identify due, for example, to the substantially similar structures of the different JNK isoforms. Thus, a need exists to identify specific cellular targets involved in rheumatoid arthritis such that agents specific for the cellular targets can be identified and used to treat patients suffering from rheumatoid arthritis. The present invention satisfies this need and provides additional advantages.

### SUMMARY OF THE INVENTION

The present invention is based on the identification of protein complexes involved in the c-Jun N-terminal kinase (JNK) signal transduction pathway in synoviocytes of subjects with arthritis, particularly inflammatory arthritis such as rheumatoid arthritis (RA) and osteoarthritis (OA). The protein complexes include a protein complex formed by the specific interaction of a mitogen activated protein (MAP) kinase kinase kinase (MAP3K), including MEKK1, MEKK2, or TAK1, and a MAP kinase kinase (MKK), including MKK4 or MKK7 (e.g., a MEKK1/MKK4, MEKK2/MKK4, or MEKK2/MKK7 complex), wherein the protein interactions can result in phosphorylation and activation of the MKK4 or MKK7, respectively; and a protein complex formed due to the specific interaction of a JNK and a MKK (e.g., a JNK/MKK4, JNK/MKK7, or JNK/MKK4/MKK7 complex), which can further comprise a JNK interacting protein (JIP) such as JIP-1 or JIP-3, wherein the protein interactions can result in phosphorylation and activation of JNK, which can further propagate the signal and result in gene expression. Accordingly, the present invention relates to a method of identifying an agent that modulates JNK mediated signal transduction in a synoviocyte.

A method of identifying an agent that modulates JNK mediated signal transduction in a synoviocyte can be performed, for example, by contacting the synoviocyte, or an extract of a synoviocyte with a test agent; and detecting a change in the formation or activity of the protein complex, for example, a protein complex comprising JNK/MKK4, JNK/MKK7, JNK/MKK4/MKK7, JNK/MKK4/MKK7/JIP-3, MEKK1/MKK4, MEKK1/MKK7, MEKK2/MKK4, MEKK2/MKK7, TAK1/MKK4, or TAK1/MKK7, or a combination thereof, in the presence of the test agent as compared to the formation or activity of the protein complex in the absence of the test agent. The synoviocyte can be a synoviocyte that is obtained from a subject, which can be any vertebrate subject such as a mammalian subject, particularly a human subject, for example, a human subject with an arthritis such as RA or OA. If desired, the synoviocyte can be contacted with a cytokine (e.g., interleukin-1) either prior to, substantially simultaneously with, or following contact of the synoviocyte with the test agent. A change in the formation of activity of the protein complex in the presence of the test agent can be detected, for example, by detecting a reduced level of formation or activity protein complex, or by detecting disruption of the protein complex. A JNK signalsome can be particularly effective in activating JNK because both MKK4 and MKK7 are maintained in proximity and function synergistically in activating JNK. As such, an agent that modulates the formation or activity of a JNK signalsome can be particularly useful for modulating JNK mediated signal transduction. The invention also provides an isolated agent that modulates JNK signal transduction activity in a synoviocyte, wherein the agent is identified by a method of the invention.

The present also invention relates to methods of identifying an agent that modulates JNK mediated signal transduction. In one embodiment, a method of the invention is performed by contacting a JNK polypeptide, and a MAP kinase kinase (MKK), particularly MKK4, MKK7, or both MKK4 and MKK7, and, optionally, a scaffold protein such as a JIP, for example, JIP-3, under conditions suitable for JNK/MKK (or JNK/MKK/JIP) protein complex formation or activity, and a test agent; and detecting a change in JNK/MKK protein complex formation or activity in the presence of the test agent as compared to the JNK/MKK protein complex formation or activity in the absence of the test agent, thereby detecting an agent that modulates JNK mediated signal transduction. In another embodiment, a method of the invention is performed by contacting an MKK polypeptide, particularly MKK4, MKK7, or both MKK4 and MKK7, and a MAP kinase kinase kinase (MAP3 K), particularly MEKK1 , MEKK2, or TAK1, under conditions suitable for MAP3K/MKK protein complex formation or activity, and a test agent; and detecting a change in MAP3K/MKK protein complex formation or activity in the presence of the test agent as compared to the MAP3K/MKK protein complex formation or activity in the absence of the test agent, thereby detecting an agent that modulates JNK mediated signal transduction. Accordingly, an agent obtained using a method of the invention is provided, wherein the agent can modulate JNK mediated signal transduction, as is a medicament comprising the agent, the medicament being useful, for example, for treating a subject with an arthritis.

The JNK polypeptide can be a JNK1, JNK2, or JNK3 polypeptide, generally a JNK1 or JNK2 polypeptide, and particularly JNK2. The JNK/MKK protein complex can be a JNK/MKK4 complex, JNK/MKK7 complex, or JNK/MKK4/MKK7 complex, the latter of which is referred to as a JNK signalsome, and which can further comprise a scaffold protein, for example, a JIP, particularly JIP-3. The JNK, MKK and test agent can be contacted in any order, as desired. Thus, a screening assay of the invention can be performed, for example, by first contacting the JNK and MKK polypeptides (and scaffold protein, where present), such that a JNK/MKK protein complex can form, then contacting the JNK and MKK polypeptides, including the JNK/MKK protein complex, with the test agent. The screening assay also can be performed, for example, by first contacting the JNK polypeptide and the test agent, or the MKK polypeptide(s) and the test agent, then contacting those components with the MKK or JNK polypeptide, respectively. In addition, the screening assay can be performed by substantially simultaneously contacting the JNK and MKK polypeptides and the test agent. Furthermore, the method can further include contacting the JNK and MKK polypeptides (and, where present, scaffold protein) with a MAP kinase kinase kinase (MAP3K), particularly MEKK1, MEKK2 or TAK1, under conditions suitable for MAP3K/MKK protein complex formation or activity.

A change in JNK/MKK protein complex formation or activity in the presence of the test agent as compared to the absence of the test agent (or in the presence of a control agent such as a molecule that has been determined not to have an ability to change JNK/MKK protein complex formation or activity) can be detected by detecting a reduced level of the JNK/MKK protein complex formation or activity; or by detecting disruption of the JNK/MKK protein complex; or by detecting an increased level in the JNK/MKK protein complex formation or activity; or by detecting stabilization of the JNK/MKK protein complex; or by detecting a reduced level of MKK phosphorylation, JNK phosphorylation, or both MKK phosphorylation and JNK phosphorylation; or by detecting a reduced level of c-Jun polypeptide phosphorylation. Where the screening assay is performed using a cell based assay, a change in JNK protein complex formation or activity in the presence of the test agent also can be detected by detecting a change in translocation of the JNK/MKK protein complex in the cell. A change in JNK/MKK protein complex formation or activity can be detected in any of various ways, including, for example, by detecting lower (or higher) levels of the complex in the presence of the test agent as compared to the absence using a method such as an immunoprecipitation assay, or by detecting lower (or higher) levels of phosphorylation of a substrate for the MAP3K (e.g., MKK4), MKK (e.g., JNK2) or JNK (e.g., c-Jun) using a kinase assay.

A test agent examined according to a method of the invention can be a peptide; a peptidomimetic; a polynucleotide; a derivative of a peptide or polynucleotide such as a peptide nucleic acid, which is a nucleic acid molecule containing one or more peptide bonds linking the nucleotide monomers; a small organic molecule such as a peptidomimetic, or any type of molecule generally considered useful or potentially useful as an agonist or antagonist of a biochemical reaction, including molecules generally useful as therapeutic agents. In addition, a test agent can be one type of molecule of a library of molecules (test agents), including, for example, a randomly generated library, a biased library, or a variegated library, which can be based on a particular molecule but varied in one or a few reactive or potentially reactive groups. The methods of the invention are particularly useful for screening a library of molecules because the methods can be conveniently adapted to a high throughput format, thus allowing, for example, a large number of different test agents, which can, but need not, be chemically related, to be examined in parallel.

A method of the invention can be performed in vitro using substantially purified components, including JNK and MKK polypeptides (or using cell extracts that contain JNK and/or MKK polypeptides), one or more test agents, a scaffold protein (e.g., a JIP) and/or MAP3K, if desired, and appropriate buffers, salts, and, where appropriate, other reagents, for example, a phosphate transfer group such as provided by ATP or a substrate such as c-Jun, and the like. A method of the invention also can be performed using a cell that expresses JNK and MKK (and a JIP and/or MAP3K, if desired), wherein the cell is contacted with the test agent. The cell generally is a vertebrate cell, particularly a mammalian cell such as a human cell. For example, the cell can be a synoviocyte, which can be from a subject suffering from an arthritis, including an inflammatory arthritis such as RA, OA, and the like, or can be a cytokine stimulated synoviocyte.

Where a method of the invention is performed in a cell, the JNK and MKK, and the JIP and/or MAP3K, when present, or any combination thereof, can be endogenous to the cell, or can be heterologous polypeptides, which can be expressed from a polynucleotide introduced into the cell under conditions such that the polypeptide can be expressed. Furthermore, the cell can be a cell in culture, including a cell of a cell line that has been adapted to passage is tissue culture, or a cell that is removed from a subject and contacted ex vivo. In one embodiment, a method of the invention is performed using a fibroblast-like synoviocyte (FLS), or an extract thereof. As disclosed herein, MAP3K/MKK and/or JNK/MKK protein complexes, including JNK signalsome formation, occur upon contact of FLS, particularly FLS from a subject with an arthritis such as rheumatoid arthritis (RA), with a cytokine such as IL-1. As such, conditions suitable for formation of the JNK/MKK and/or MAP3K/MKK protein complexes include contacting such FLS with a cytokine. Accordingly, the methods of the invention can be performed, for example, by contacting the cells with a cytokine such as IL-1, then further contacting the cells, or an extract of the cells with a test agent, or by contacting the cells with a test agent, then further contacting the cells with the cytokine. The methods of detecting a change in JNK mediated signal transduction can be performed in the cells, or using an extract of the cells.

The present invention also relates to a method of ameliorating an arthritis in a subject, wherein the arthritis is associated with cells exhibiting abnormally high JNK signalsome mediated signal transduction. Such a method can be performed, for example, by contacting the cells of the subject with an agent that reduces or inhibits JNK/MKK and/or MAP3K/MKK protein complex formation or activity, thereby reducing or inhibiting JNK signalsome mediated signal transduction in the cells and ameliorating the arthritis. For example, the arthritis can be RA, and the cells exhibiting abnormally high JNK signalsome mediated signal transduction can be RA synoviocytes.

According to a method of the invention, cells of the subject can be contacted with the agent by administering the agent to the subject, for example, systemically such that the agent circulates to the cells, to directly to one or more sites of the arthritis (and the cells) in the subject. Cells of the subject also can be contacted with the agent ex vivo, then the cells that have been contacted with the agent can be administered back into the subject to the subject, for example, to a site of the arthritis in the subject.

The present invention also relates to an isolated protein complex. In one embodiment, the protein complex is a JNK/MKK protein complex, for example, a JNK2/MKK4 protein complex, or a JNK2/MKK7 protein complex, which is isolated from synoviocytes, particularly cytokine stimulated synoviocytes and/or arthritis synoviocytes. In another embodiment, the protein complex is a JNK signalsome, comprising a JNK polypeptide, a MKK4 polypeptide and an MKK7 polypeptide. In still another embodiment, the JNK signalsome comprises a scaffold protein such as a JIP, particularly JIP-3. In still another embodiment, the protein complex is a MAP3K/MKK protein complex, for example, an MEKK2/MKK4 protein complex or an MEKK2/MKK7 protein complex. The present invention further relates to an antibody that specifically binds a protein complex of the invention, wherein the antibody does not substantially bind an isolated JNK, MKK, JIP, or MAP3K polypeptide. In one embodiment, the antibody specifically binds a JNK signalsome.

### BRIEF DESCRIPTION OF THE DRAWINGS

FIG. 1 illustrates MAPK family members and pathways in which the kinases are believed to be involved. The first column illustrates a generalized kinase based signal transduction pathway, beginning with a signal and ending with activation of transcription factors. The second column illustrates an ERK pathway; the third column illustrates a JNK pathway; and the fourth and fifth columns illustrate two arms of a P38 pathway.

FIG. 2 illustrates various JNK protein complexes (JNK shown as solid dark box), including JNK/MKK7, JNK/MKK4, and the JNK signalsome (JNK/MKK4/MKK7). Positions of phosphorylated threonine (Thr) and tyrosine (Tyr) amino acid residues involved in regulation of the kinase activity are shown, as are serine 63 and serine 73 of the c-Jun transcription factor. As shown, activation of c-Jun results, in part, in the expression of matrix metalloproteinase (MMP) genes such as collagenase 1 and collagenase 3.

### DETAILED DESCRIPTION OF THE INVENTION

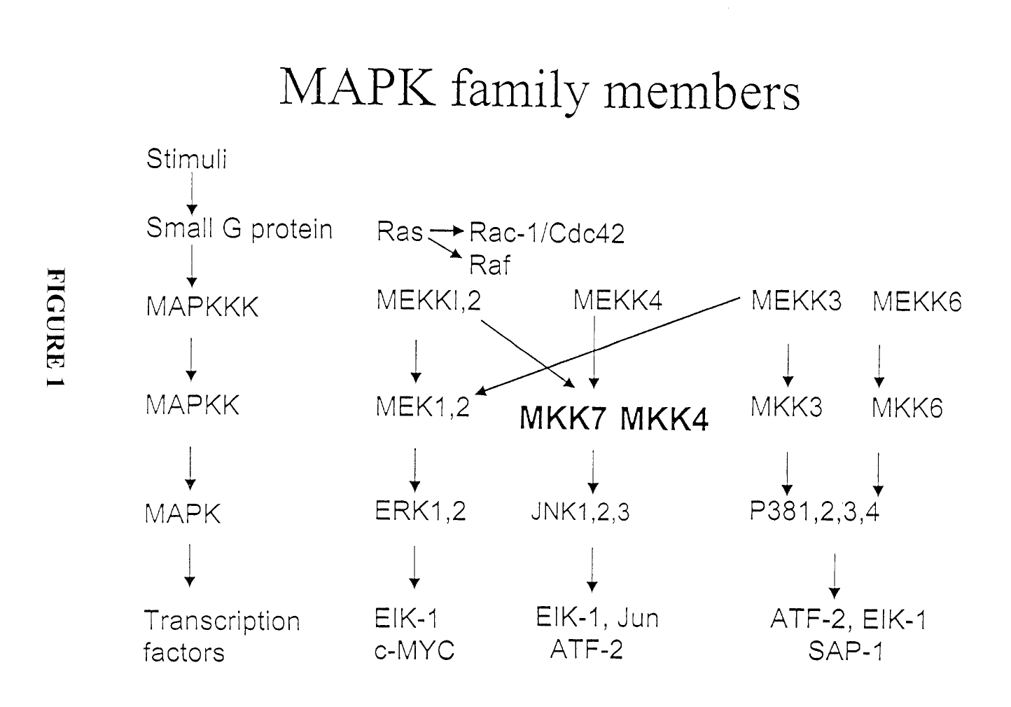

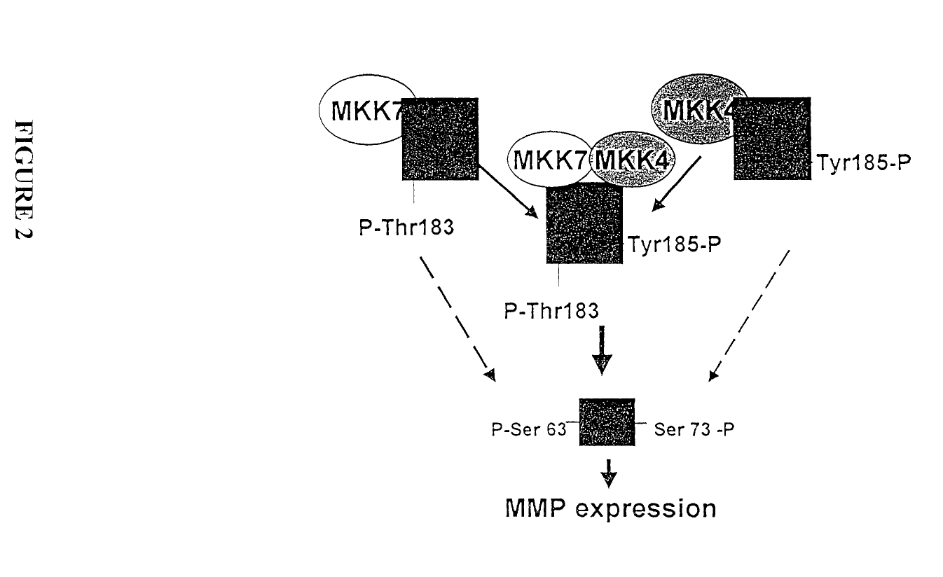

The present invention is based on the identification of upstream components of the c-Jun N-terminal kinase (JNK) signal transduction pathway in synovial cells in subjects with an inflammatory arthritis, including, for example, rheumatoid arthritis (RA) and osteoarthritis (OA), and on the identification of specific protein complexes, including the identification of JNK/MKK (mitogen activated protein (MAP) kinase kinase) protein complexes such as the JNK signalsome, which is activated in synovial cells in arthritis and mediates cytokine stimulated phosphorylation of c-Jun and c-Fos, which, together, form the transcription factor, A1; and MAP kinase kinase kinase (MAP3K)/MKK protein complexes, which generate phosphorylated MKKs that can form protein complexes to JNK and activate JNK. As disclosed herein, JNK specifically associates with either of or both of two MKKs, MKK4 and MKK7, to form JNK/MKK4 and JNK/MKK7 complexes, respectively, or a JNK signalsome, which comprises JNK, MKK4 and MKK7, and can further comprise a scaffold protein such as a JNK interacting protein (JIP), in RA synoviocytes and OA synoviocytes (see FIGS. 1 and 2). As further disclosed herein, the formation of the JNK complexes is induced in arthritis synoviocytes due to phosphorylation of the MKK polypeptides by a MAP3K, particularly MEKK1, MEKK2, or TAK1.

Accordingly, the present invention provides methods of identifying agents that can selectively modulate the JNK mediated signal transduction in cells in arthritis patients, including, for example, agents that can selectively reduce or inhibit the formation of a JNK/MKK protein complex such as a JNK/MKK4 complex, or of a MAP3K/MKK protein complex such as MEKK2/MKK4. In addition, the present invention provides agents that can selectively modulate JNK signal transduction activity, including, for example, agents that can selectively reduce or inhibit JNK signalsome mediated signal transduction in cells of a subject suffering from an arthritis such as RA, OA, and the like, and further provides methods of ameliorating arthritis, including preventing or reducing bone and/or cartilage damage in joints, in a subject by administering such an agent to an arthritis patient such that JNK signalsome mediated signal transduction in synovial cells of the subject is reduced or inhibited.

Despite increased understanding of the aggressive characteristics of arthritis fibroblast-like synoviocytes (FLS), there has been little information on the signal transduction pathway(s) that lead to expression of the factors involved in joint destruction. Activation of nuclear factor kappa B (NF-κB) by the inhibitor of NF-κB (IκB) kinases and the potential role of MAP kinases has been examined, for example, by gene therapy studies using dominant negative IκB kinase constructs. The results of these experiments suggest that NF-κB plays a central role in synovial inflammation, though it is less important in matrix degradation.

Dissecting the role of MAP kinases has been hampered by the lack of selective reagents that inhibit JNK, although effective inhibitors of p38 and ERK, which also are activated by MAP kinases, are available (SB203580 and PD98059, respectively). The potential importance of JNK in RA was suggested by studies demonstrating that cytokines such as interleukin-1 (IL-1) and tumor necrosis factor-alpha (TNFα) lead to much more efficient phosphorylation of JNK in RA FLS than in cells derived from osteoarthritis (OA) synovium (Zheng and Guan, J. Biol. Chem. 268:23933-23939, 1993). These results led to a hypothesis that activation of JNK contributes to destruction of cartilage and bone in RA, and that, since JNK blockade can be effective in this condition, an upstream inhibitor could be effective at lower concentrations and without requiring inhibition of multiple isoforms of JNK.

The basis of this hypothesis lies in an understanding of the key transcription factors involved in MMP expression in rheumatoid synovitis. Activator protein-1 (AP-1) is of particular interest due to the promiscuous distribution of its binding site among the upstream regulatory regions of MMP genes, including, for example, collagenase-1, collagenase-3, and stromelysin-1. AP-1 is a protein complex that includes the c-Jun and c-Fos proto-oncogenes; the heterodimer is held together by a leucine zipper, in which residues of leucine interact with the homologous region on its binding partner. Heterodimers and homodimers of the Jun and Fos family proteins interact with specific binding sites in the regulatory regions of genes, thereby inducing increased mRNA transcription.

Immunoreactive AP-1 has been detected in human RA synovium, and c-Jun is highly expressed in the intimal lining (Asahara et al., Arthritis Rheum. 40:912-918, 1997). AP-1 binding, as detected by electrophoretic mobility shift assays (EMSA), also is elevated in RA synovium (see Fujisawa et al., Arthritis Rheum. 39:197-203, 1996; Asahara et al., Mol. Biol. Internatl. 37:827-832, 1995; Han et al., Autoimmunity 28:197-208, 1998). In contrast, OA synovium was reported to have little or no AP-1 binding activity. Transcription factor activation also has been evaluated in animal models of arthritis. Decoy AP-1 oligonucleotides inhibited joint destruction in murine collage-induced arthritis (CIA; Shiozawa et al., J. Clin. Invest. 99:1210-1216, 1997). To characterize the kinetics of transcription factor activation and subsequent MMP gene expression in inflammatory arthritis, northern blot analysis and EMSA was performed on samples obtained from the joints of mice with collagen-induced arthritis. AP-1 expression increased in joint tissue before the onset of clinical arthritis and persisted as the disease progressed (see Han et al., supra, 1998; Boyle et al., J. Pharmacol. Exp. Ther. 296:495-500, 2001, each of which is incorporated herein by reference).

MAP kinases play an important role in AP-1 activation by phosphorylating AP-1 subunits at specific amino acid residues. c-Jun is phosphorylated at two N-terminal serine residues (amino acids 63 and 73) by at least three closely related JNK proteins, JNK1, JNK2 and JNK3, which exist as multiple isoforms generated by alternative splicing. JNK2 binds c-Jun with at least 25-fold higher affinity than JNK1 and is likely the more physiologically relevant activator of AP-1 (Kallunki et al., Genes Devel. 9:2996-3007, 2001). JNK3 appears to be less important in arthritis because it is primarily expressed in neural tissue. The N-terminal phosphorylation of c-Jun by JNK enhances the transcriptional activity about 30-fold, and transcriptional activity is further amplified by increased c-jun gene transcription. Cytokine stimulation also induces c-jun and c-fos mRNA accumulation in cultured FLS (Boyle et al., Arthritis Rheum. 40:1772-1779, 1997). These results indicate that modification of AP-1 is responsible for both constitutive and inducible gene expression, with JNK playing a critical role activating c-Jun during the inflammatory response.

The signal transduction pathways that lead to activation of AP-1 are complex and can involve the MAP kinases. This diverse group of kinases responds to inflammatory stimuli, and comprise parallel protein kinase cascades. Three major MAP kinase families have been identified. In addition to the JNK proteins (also called stress activated protein kinases, or SAPK), there are two other well-defined pathways—the extracellular signal-regulated kinases 1 and 2 (ERK1/ERK2; also referred to as p42/p44 MAPKs); and the p38 MAP kinases (see FIG. 1).

Each MAP kinase family is phosphorylated and activated by a cascade of specific kinases, MAP kinase kinases (MKKs; also referred to as MEKs). In general, this kinase cassette includes enzymes in series (from MAPKKK to MAPKK to MAPK) that serve as an “on-off” switch for the particular MAPK (see FIG. 1). However, controversy remains as to the specificity of each enzyme and the degree of promiscuity for substrates, although some patterns of activation have been defined. For instance, MKK1 and MKK2 primarily activate ERKs (Zheng and Guan, supra, 1993; Wu et al., Proc. Natl. Acad. Sci., USA 90:173-177, 1993), while MKK3 and MKK6 selectively activate the p38 kinases (see, for example, Raingeaud et al., Mol. Cell Biol. 16:1247-1255, 1996; Han et al., J. Biol. Chem. 271:2886,2891, 1996. MKK4 (also called JNKK1, and SEK) activates both p38 and JNK (Lin et al., Science 268:286-290, 1995, which is incorporated herein by reference), whereas the more recently described MKK7 (JNKK2) appears to be more specific for the JNK pathway (Tournier et al., Proc. Natl. Acad. Sci., USA 94:7337-7342, 1997; Moriguchi et al., EMBO J. 16:7045-7053, 1997, each of which is incorporated herein by reference). Prior to the present disclosure, however, the relative contribution of each upstream kinase to mediators of the joint destruction associated with rheumatoid arthritis remained undefined.

The activation of ERK, p38, and JNK in cultured FLS was examined to identify a role of MAP kinases in collagenase gene expression and joint destruction in arthritis (Han et al., J Pharmacol. Expt. Ther. 291:124-130, 1999, which is incorporated herein by reference). All three kinase families were constitutively expressed in synoviocytes, and cytokines such as IL-1 led to rapid phosphorylation of p38 and ERK in both RA FLS and OA FLS. JNK activation was significantly greater in RA FLS as compared to OA cells, and the increased JNK activation correlated with higher collagenase gene expression in cultured RA cells. Activation of JNK also was greater in RA cells stimulated with TNFα. The differences between RA and OA were not due to variations in cytokine receptor density among cells in the different diseases, as unrelated stimuli such as anisomycin and phorbol esters activated JNK in both RA and OA FLS (Han et al., supra, 1999).

JNK was identified as the key MAPK involved in the induction of MMP genes in RA FLS (Han et al., supra, 1999). Low concentrations of the selective p38 inhibitor, SB203580, which completely inhibit p38 function, had little or no effect on IL-1-induced collagenase expression, AP-1 activation, or c-jun expression. Similarly, PD98059, which inhibits MEK½ and blocks ERK activation, only modestly decreased collagenase mRNA accumulation. Because no selective JNK inhibitor was available at the time those studies were conducted, high concentrations of SB203580 were used as to block JNK (see Cuenda et al., FEBS Lett. 364:229-233, 1995). Although 25-50 μM of SB203580 inhibited certain splice variants of JNK2, these concentrations blocked p38 as well as other kinases, including c-raf, that could alter cellular function (De Laszlo et al., Bioorg. Med. Chem. Lett. 8:2689-2694, 1998). Thus, while SB203580 is a nonspecific tool for evaluating JNK function, high concentrations of SB203580 significantly decreased collagenase gene expression and AP-1 activation.

The JNK inhibition results were extended using the first available specific JNK inhibitor, SP600125, which inhibits JNK1 and JNK2 with an IC50 of about 40 nM; SP600125 is less potent on JNK3 (IC50=90 nM), but this isoform is expressed only in neural tissue, and does not inhibit p38, ERK or a variety of other kinases (Han et al., J. Clin. Invest. 108:73-81, 2001, which is incorporated herein by reference). SP600125 inhibited IL-1-induced c-Jun phosphorylation, c-jun mRNA induction and collagenase gene expression in FLS, but did not inhibit the basal levels of collagenase gene expression or c-Jun phosphorylation in cultured FLS. These results confirmed a central role for JNK in the regulation of cytokine-induced MMPs in FLS, and indicated that JNK is a potential target for chondroprotective therapy.

The JNK pathway is considerably more efficient than either p38 or ERK as an enabler of AP-1 mediated gene transcription and can contribute to increased MMP production in arthritis FLS. Since JNK also regulates cytokine gene expression, including TNFα, MAP kinase profiles in arthritis cells, particularly inflammatory arthritis cells, can be indicative of the phenotype of arthritis FLS. As disclosed herein, and in contrast to other cell types, the MAP kinase kinase MKK4 specifically associates with JNK to form a functional JNK/MKK4 complex in arthritis cells, including RA and OA cells (Example 1), which can further specifically associate with MKK7 to form a functional JNK signalsome (see FIG. 2). In addition, the MAP3Ks, MEKK1, MEKK2 and TAK1, specifically associate with and phosphorylate MKK4 and MKK7 in arthritis synoviocytes. As such, the present invention defines upstream components of the JNK signal transduction pathway in synoviocytes, including arthritis synoviocytes such as RA and OA synoviocytes, and cytokine-stimulated synoviocytes. These upstream components of the JNK signal transduction pathway include MKK4 and MKK7, which are upstream of, form a complex with, and activate JNK; and MEKK1, MEKK2, and TAK1, each of which is upstream of, forms a complex with, and activates MKK4 and MKK7.

Of the signal transduction pathways that regulate MMP expression, the MAPKs play a central role. All three MAPK families (ERK, JNK, and p38) can regulate collagenase gene expression (Megenshol et al., Nucl. Acids Res. 29:4361-4372, 2001). The relative hierarchy of the individual MAPKs is dependent on the cell type and inflammatory stimulus. In FLS, the central role of JNK on the expression and regulation of collagenase-1 is related to its ability to phosphorylate the c-Jun component of the transcription factor AP-1, a key regulatory molecule in MMP gene transcription (Han et al., supra, 2001). The present study focused on upstream activators of JNK in FLS in order to characterize the pathways involved in JNK activation in arthritis.

The MAPK enzymes are regulated by the upstream MKKs, including MKK4 and MKK7, which are known to phosphorylate JNK. Like other MAPK family members, JNK is activated by phosphorylation of threonine and a tyrosine residue in the TXY motif by the dual specific MAPK kinases (Fleming et al., Biochem. J. 352:145-154, 2000). Previous studies suggested that MKK4 and MKK7 phosphorylate JNK on the Tyr and Thr residues, respectively, and that two kinases have a synergistic effect in their ability to activate JNK (Lawler et al., Curr. Biol. 8:1387-1390, 1998). MKK7 appears to be primarily activated by cytokines such as TNFα and IL-1, whereas MKK4 is primarily activated by environmental stress in some in vitro systems (Davis, Cell 103:239-252, 2000).

As disclosed herein, immunohistochemistry studies demonstrated that both MKK4 and MKK7 are expressed in the intimal lining and sublining region of synovial tissue (Example 1). No differences were observed between RA and OA synovium with respect to the amounts of MKK proteins. To evaluate the activation state of synovial MKK4 and MKK7, phosphospecific antibodies were used for immunohistochemistry and western blot analysis. Phospho-MKK4 and phospho-MKK7 expression were identified in RA synovium and in OA synovium, particularly in the intimal lining. Western blot studies confirmed this result by identifying abundant immunoreactive total and phospho-MKK4 and phospho-MKK7 in RA synovium. These results indicate that MKK4 and MKK7, which are upstream of JNK, are activated in arthritis synoviocytes, are localized to regions where AP-1 proteins and MMPs are expressed, and can phosphorylate JNK (Example 1).

The observation that activated MKK4 and MKK7 protein expression is especially high in the synovial intimal lining indicated that the fibroblast-like cells in the synovium (FLS) could express the kinases. As such, regulation of MKK4 and MKK7 was examined in cultured FLS. MKK4 and MKK7 expression were readily demonstrated in RA and OA FLS by western blot analysis (Example 1). Both kinases were rapidly phosphorylated after IL-1 stimulation, and no differences were observed between FLS from different diseases. These results are in contrast to those obtained using murine embryonic fibroblasts, in which only MKK7 (but not MKK4) was activated by cytokines (Tournier et al., Genes Devel. 15:1419-1426, 2001). These results indicate that the regulation of MKK4 varies with cell type and culture conditions, and that cytokine stimulation can result in a synergistic activation of JNK through via MKK4 and MKK7 in FLS.

Signal transduction proteins can form stable complexes in cells, thereby orchestrating and facilitating the specific activation pathways. Signaling specificity for the MAPK can be mediated, in part, through the formation of kinase units that influence their localization, specificity and targets. These complexes can involve the interaction between kinase components of signaling modules or interactions with substrates or scaffold proteins (Davis, supra, 2000). For example, the ERK activators MEK1 and MEK2 contain unique conserved sequences in the N-terminus that specifically bind ERK½ (Whitmarsh et al., Science 281:1671-1674, 1998). This docking site in MEK1 is required for ERK2 activation (Xu et al., J. Biol. Chem. 274:34029-34035, 1999). Other interactions have been described in ERK pathway, including complexes between MEK1-Ras and Raf-1. Scaffold proteins have been identified in MAPK signal transduction cascade, such as MP-1 which interacts with ERK-1 and MEK1.

Similar complexes have been described for JNK pathway. For example, JNK interacting protein-1 (JIP-1) and JIP-2 are closely related proteins that bind to JNK, MKK7, and mixed lineage protein kinases (Whitmarsh et al., supra, 1998). Another scaffold protein called JNK/SAPK associated protein-1 (JSAP-1), an alternatively spliced variant of JIP-3, also may interact with JNK, MKK4, and MEKK1 (Ito et al., Mol. Cell Biol. 19:7539-7548, 1999). As discussed above, studies with murine embryonic fibroblasts demonstrated that MKK7, but not MKK4, forms a stable complex with JNK (Tournier et al., supra, 2001). In contrast to the embryonic cells, JNK in FLS forms a novel signalsome that contains JNK as well as its two upstream kinases MKK4 and MKK7 (Example 1). The components of the complex were defined by a series of immunoprecipitation experiments, where all three kinases co-precipitated with antibodies directed at any individual component (Example 1). JIP-1 was not a component of the signalsome, although a separate JIP-1/MKK7/JNK complex also may exist in FLS; however, JIP-3 was detected in the complex and likely represents a scaffold protein for the tri-kinase signalsome.

Laser confocal microscopy confirmed that MKK4, MKK7 and JNK co-localize in the cytoplasm of resting cells, and also demonstrated that the complex migrated to the nucleus after IL-1 stimulation. MKK7 has been shown to accumulate in the nucleus in stressed cells (Merritt et al., J. Biol. Chem. 274:10195-10202, 1999). Phosphorylated JNK also accumulates in the nucleus, suggesting that the kinase unit interacts with its substrates in this site. Nuclear localization of MKK4 and MKK7 contrasts with the cytoplasmic location caused by nuclear export of ERK activators MEK1 and MEK2 (Fukuda et al., J. Biol. Chem. 272:32642-32648, 1997). Inactive p38 MAPK also is located in the nucleus and is rapidly exported to the cytoplasm after activation, perhaps due to its nuclear substrate, which is also exported from the nucleus following activation(Ben-Levy et al., Curr. Biol. 8:1049-1057, 1998).

The mechanism of nuclear migration by MKK4 and MKK7 is uncertain because these kinases do not include an obvious nuclear localization sequence. The absence of an nuclear exclusion site may permit the localization of MKK4 and MKK7 in the nucleus, or a nuclear localizing sequence in JNK may escort the tethered MKKs to the nucleus. However, in some circumstances, the ability of MKK7 to localize to the nucleus does not necessarily correlate with downstream JNK activation. For example, in cells expressing a mutant MKK7 that is excluded from the nucleus, JNK was efficiently activated and accumulated in the nucleus (Tournier et al., Mol. Cell Biol. 19:1569-1581, 1999). These observations, together with the results disclosed herein, indicate that MKK7 (and possibly MKK4) activate JNK in the cytoplasm, and that the activated JNK signalsome redistributes from the cytoplasm to the nucleus.

Regardless of the mechanism of translocation, the JNK signalsome disclosed herein is functionally active. For example, kinase assays on the immunoprecipitated complexes using GST-c-Jun as a substrate revealed that IL-1 stimulation markedly increased the capacity of the precipitate to phosphorylate GST-c-Jun, regardless of whether anti-MKK4, anti-MKK7, or anti-JNK antibodies were used to isolate the signalsome. That JNK was the enzyme in the complex that phosphorylated GST-c-Jun was confirmed using the specific JNK inhibitor SP600125, which ablated the ability of the immunoprecipitates to phosphorylate the substrate (Example 1).

Although JNK inhibition can decrease bone destruction in arthritis (Han et al., supra, 2001; see, also, Example 2), methods for generally inhibiting JNK are not particularly useful because JNK mediates several signal transduction pathways in various cell types and because the similarities of the various JNK isoforms makes it difficult to identify agents that are selective for a particular isoform. The identification of specific protein complexes in synoviocytes of arthritis patients, including the JNK/MKK4 complex, JNK/MKK7 complex, and the JNK signalsome (JNK/MKK4/MKK7 complex), which can further include scaffold proteins such as a JIP, for example, JIP-3, as well as the MAP3K/MKK complexes, MEKK1/MKK4, MEKK1/MKK7, MEKK2/MKK4, MEKK2/MKK7, TAK1/MKK4 and TAK1/MKK7, thus provides targets that can be utilized in screening assays to identify agents that selectively modulate JNK mediated signal transduction in cells of arthritis patients and, therefore, provides a means to identify agents that can be useful for ameliorating the signs and symptoms associated with the arthritis.

As used herein, the term “specific interaction”, “specifically binds”, “specifically associates”, or the like means that two or more molecules form a complex that is relatively stable under physiologic conditions. The term is used herein in reference to various interactions, including the interaction of a JNK polypeptide such as JNK1, JNK2 or JNK3 with either or both of MKK4 and/or MKK7 to form a protein complex; the interaction of the intracellular components of a JNK signal transduction pathway such as the interaction of MEKK1, MEKK2, or TAK1, and MKK4 or MKK7 to form protein complexes; and the interaction of a scaffold protein such as a JIP, particularly JIP-3, with protein components of the JNK signalsome; as well as the interaction of a specific antibody and its antigen. For convenience, the term “JNK/MKK protein complex” is used herein to refer to a complex formed due to the specific association of a JNK and at least one MKK polypeptide, particularly MKK4 and/or MKK7, and which can further include a scaffold protein such as JIP-3, including, for example, a complex of JNK, MKK4 and MKK7. The term “JNK signalsome” is used herein to refer more specifically to a JNK/MKK protein complex comprising JNK, MKK4, and MKK7, and which can further include, for example, JIP-3. The term “MAP3K/MKK protein complex” is used herein to refer to a complex formed due to the specific association of a MAP3K polypeptide, particularly MEKK1, MEKK2, or TAK1, and an MKK polypeptide, particularly MKK4 and MKK7.

A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as in a cell in a human or other vertebrate or invertebrate, as well as conditions that occur in a cell culture such as used for maintaining mammalian cells or cells from another vertebrate organism or an invertebrate organism. A specific interaction such as that occurring among the components of a JNK/MKK protein complex such as a JNK signalsome also is generally stable under in vitro conditions that mimic physiologic conditions, including reaction mixtures having an appropriate buffer capacity and pH, salt concentration, metal ion concentration, or the like. Conditions suitable for the protein complex formation or activity also can include the presence of one or more substrates, including, for example, a molecule comprising a phosphate donor group such as ATP or GTP, or a molecule comprising a phosphate acceptor group such a c-Jun protein, which can be phosphorylated by JNK.

As used herein, the term “JNK mediated signal transduction” refers to an intracellular pathway that includes phosphorylation of JNK to an activated form, such that it can phosphorylate c-Jun on serine 63 and serine 73. For purposes of the present invention, the JNK mediated signal transduction pathway as defined in cytokine stimulated FLS is of particular interest, and includes a MAP3K, particularly MEKK2, which specifically associates with, phosphorylates, and activates a MKK, particularly MKK4 and/or MKK7; and MKK4 and MKK7, which specifically associate with, phosphorylate, and activate JNK, which, in turn, can phosphorylate c-Jun at serine 63 and serine 73; the phosphorylated c-Jun can then interact with another c-Jun polypeptide or with a c-Fos polypeptides to form an active transcription factor such as AP-1, which induces expression of various genes, including, for example, collagenase genes (see FIG. 1). Such JNK mediated signal transduction requires, in part, the formation of a JNK signalsome, i.e., a JNK/MKK4/MKK7 protein complex, and, therefore, is further referred to herein as “JNK signalsome mediated signal transduction”.

The present invention provides screening assays useful for identifying agents that increase or decrease the formation and/or activity of such MAP3K/MKK and/or MKK/JNK protein complexes, particularly in FLS, including cytokine stimulated FLS and arthritis FLS such as RA FLS and OA FLS, thus providing a means to modulate JNK mediated signal transduction in the FLS. In particular, the screening assays can identify agents that alter the formation and/or activity of a complex comprising a MEKK2 MAP3K and an MKK4 or MKK7 MKK, or a complex comprising an MKK4 and/or MKK7 MKK and a JNK1, JNK2 or JNK3 JNK, including a JNK signalsome, or a complex comprising a scaffold protein such as JIP-3 and one or more components of a JNK signalsome, and, therefore, provides a means to identify agents that can be useful for ameliorating an arthritis such as rheumatoid arthritis.

As used herein, the term “modulates,” when used in reference to JNK mediated signal transduction, means that the level of signal transduction is reduced (or inhibited) or is increased due to an effect of an agent on the formation or activity of a protein complex comprising a JNK polypeptide and a MKK, particularly MKK4 and/or MKK7, or a protein complex comprising a MAP3K and a MKK, or a complex comprising a scaffold protein and a JNK signalsome (e.g., JNK/MKK4/MKK7/JIP-3), or a component thereof. For example, JNK signal transduction can be decreased due to an agent that can reduce or inhibit the formation of such a protein complex or that can disrupt such a complex. In comparison, JNK signal transduction can be increased due to an agent that can increase the formation or stability of such a protein complex. Such a reduction or inhibition or an increase can be detected using the methods disclosed herein, including, for example, by performing assays in parallel, wherein the samples lack or contain a test agent, or contain a control agent such as SP600125, which is known to inhibit JNK activity (Han et al., supra, 2001), or by detecting a difference in migration of JNK in an EMSA due to the presence (or absence) of the test agent as compared to the absence (or presence) of the test agent. Generally, though not necessarily, a series of samples containing varying amounts of a test agent are examined such that an effective amount for modulating the formation or activity of a JNK/MKK complex, a MAP3K/MKK complex, or a scaffold protein/JNK signalsome complex, can be identified and a dose response effect, if such a response occurs, can be detected.

An agent can act with respect to any component involved in the formation or activity of the protein complex, including, for example, on the MKK component (e.g., MKK4 and/or MKK7), on the JNK component, on the scaffold protein component (e.g., JIP-3), or on an MAP3K such as MEKK1, MEKK2 or TAK1, which contributes to formation of the complex by phosphorylating MKK4 or MKK7. For example, the agent can be a small molecule that acts by disrupting (or destabilizing) the interactions involved in such complexes, can be a dominant negative polypeptide (or peptidomimetic) that acts by competing with a normal, naturally occurring component of the complex, or can be a molecules that alters translocation of the complex to the intracellular compartment in which the complex normally acts. An agent that reduces or inhibits the formation or activity, for example, of a JNK signalsome, can be useful for reducing the expression of gene products that are regulated due to JNK activity, for example, collagenase expression in synoviocytes of an arthritis patient, thus preventing joint destruction and ameliorating the signs and symptoms of the arthritis in the subject. An agent that increases the formation of (or stabilizes) a JNK signalsome can be useful for stimulating such gene expression in synoviocytes, for example, in an experimental animal, thus providing a model system useful for studying the etiology of an inflammatory arthritis.

A screening assay of the invention can be performed, for example, by contacting a JNK polypeptide such as JNK2, and an MKK such as MKK4, MKK7, or both MKK4 and MKK7, and, optionally, a scaffold protein, for example, a JIP such as JIP-3, under conditions suitable for JNK/MKK protein complex formation or activity, and a test agent; and detecting a change in JNK/MKK protein complex formation or activity in the presence of the test agent as compared to the JNK/MKK protein complex formation or activity in the absence of the test agent, thereby detecting an agent that modulates JNK mediated signal transduction. As disclosed herein, the JNK and MKK polypeptides (and a scaffold, when present) and the test agent can be contacted in any order, as desired, and the order of addition can provide a means for identifying agents that act in various ways. For example, where the JNK and MKK polypeptides are first contacted such that a JNK/MKK protein complex can form, then the JNK/MKK protein complex is contacted with the test agent, an agent that disrupts the JNK/MKK complex can be identified by detecting a rapid decrease in the amount of complex. It should be recognized, however, that protein complex formation is a dynamic process that includes the association and dissociation of particular complexes over time. Thus, a test agent that is contacted with pre-formed JNK/MKK complexes also can act by reducing or inhibiting the ability of the JNK and MKK polypeptides to specifically associate. Such an agent can be distinguished from one that disrupts a JNK/MKK protein complex by detecting a relatively slower decrease in the level of complex with time after contact with the agent.

A screening assay of the invention can further include contacting the JNK and MKK polypeptides with a MAP3K, under conditions suitable for MAP3K/MKK protein complex formation or activity. In addition, it should be recognized that, where a test agent is being examined for the ability to increase or decrease MAP3K/MKK protein complex formation or activity, the reaction need not contain a JNK polypeptide, unless, for example, detecting a change in the MAP3K/MKK complex formation or activity requires detecting the ability of MKK to effect phosphorylation of JNK, or of JNK to effect phosphorylation of c-Jun (see, for example, Davies et al., Biochem. J. 351:95-105, 2000, which is incorporated herein by reference; see, also, Example 1).

Any method as disclosed herein or otherwise known in the art can be used to detect whether a test agent increases or decreases the formation or activity of a MAP3K/MKK protein complex, JNK/MKK protein complex or a scaffold protein/JNK signalsome protein complex. For example, methods for determining whether two or more molecules interact specifically to form a protein complex are well known and include, for example, equilibrium dialysis, surface plasmon resonance, electrophoretic mobility shift or supershift assays, immunoprecipitation assays, and the like (see Example 1; see, also, Han et al., supra, 1998, 1999). Similarly, methods for detecting the activity of MAP3K/MKK protein complex, which can phosphorylate MKK4 or MKK7, or of a functional JNK/MKK protein complex, which can phosphorylate JNK, which, in turn, can phosphorylate a c-Jun polypeptide are well known and include, for example, detecting phosphorylation of relevant amino acids of the kinase substrate (e.g., Thr-183 of JNK by MKK7 or Tyr-85 of JNK by MKK4; see FIG. 2), or by detecting expression of a reporter gene operatively linked to an AP-1 transcriptional regulatory element, or by detecting phosphorylation of serine 63 and/or serine 73 of a c-Jun polypeptide.

A change in JNK/MKK and/or MAP3K/MKK protein complex formation or activity, including a change in a scaffold protein/JNK signalsome protein complex formation or activity, can be detected as an increase or a decrease in the amount of the protein complex as determined at a particular time or for a particular period of time, or can be detected as an increase or decrease in the activity associated with the complex, for example, phosphorylation of a MKK by a MAP3K, or phosphorylation of a JNK by a MKK, or another downstream event of the JNK signal transduction pathway such as c-Jun phosphorylation by JNK or expression of a gene (or reporter gene) regulated by a transcription factor comprising c-Jun (see Example 1). As disclosed herein, the change in protein complex formation or activity is with respect to the amount or activity of the complex under control conditions, which include conditions in which no test agent is present, or conditions in which a molecule corresponding to the test agent, but known not to affect the formation or activity of the protein complex is present.

A test agent examined according to a method of the invention can be a peptide; a peptidomimetic; a polynucleotide; a derivative of a peptide or polynucleotide such as a peptide nucleic acid, which is a nucleic acid molecule containing one or more peptide bonds linking the nucleotide monomers; a small organic molecule such as a peptidomimetic, or any type of molecule generally considered useful or potentially useful as an agonist or antagonist of a biochemical reaction, including molecules generally useful as therapeutic agents. In addition, a test agent can be one type of molecule of a library of molecules (test agents), including, for example, a randomly generated library, a biased library, or a variegated library, which can be based on a particular molecule but varied at one or a few positions or with respect to one or a few reactive or potentially reactive groups.

The methods of the invention are particularly useful for screening a library of molecules because the methods can be conveniently adapted to a high throughput format, thus allowing, for example, a large number of different test agents, which can, but need not, be chemically related, to be examined in parallel. Methods for preparing a combinatorial library of molecules that can be tested for a desired activity are well known in the art and include, for example, methods of making a phage display library of peptides, which can be constrained peptides (see, for example, U.S. Pat. Nos. 5,622,699 and 5,206,347; Scott and Smith, Science 249:386-390,1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference); an oligosaccharide library (York et al., Carb. Res. 285:99-128, 1996; Liang et al., Science 274:1520-1522, 1996; Ding et al., Adv. Expt. Med. Biol. 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem. 37:1385-1401, 1994; Ecker and Crooke, BioTechnology 13:351-360, 1995; each of which is incorporated herein by reference). Small organic molecules can be particularly useful for purposes of the present invention because such molecules generally can be conveniently formulated for administration to a living subject.

Polynucleotides can be particularly useful as agents that can modulate the formation or activity of a complex comprising, for example, a JNK polypeptide and a MKK polypeptide because nucleic acid molecules having binding specificity for cellular targets, including cellular polypeptides, exist naturally, and because synthetic molecules having such specificity can be readily prepared and identified (see, for example, U.S. Pat. No. 5,750,342). A polynucleotide agent can act, for example, by binding specifically to a component of the complex (e.g., JNK) but, generally, not to the component when it is in a free form, thus providing specificity for the complex. As disclosed herein, polynucleotides useful, for example, to ameliorate RA in a subject also can be polynucleotides such as antisense molecules, ribozymes, triplexing molecules, interfering RNA molecules, or the like, which act to reduce or inhibit transcription or translation of a component of the JNK/MKK complex (e.g., a JNK signalsome), thereby reducing or inhibiting JNK mediated signal transduction.

Additionally, polypeptides, peptidomimetics, small organic molecules, and the like can be useful as agents for modulating the formation or activity of a complex comprising a JNK, MKK, scaffold protein, and/or MAP3K. Such agents can act by blocking or competing with the specific association of the components of the JNK/MKK complex, or by disrupting (or stabilizing) the complex. For example, a polypeptide, peptidomimetic, or small molecule agent can act by reducing or inhibiting the specific interaction of a JNK signalsome with a scaffold protein, thereby destabilizing the JNK signalsome. A polypeptide agent also can be a dominant negative form of a component of a MAP3K/MKK or JNK/MKK such as a dominant negative form of a JNK2, MKK4, MKK7, JIP-3, MEKK1, MEKK2 or TAK1 polypeptide. A dominant negative polypeptide agent, or a peptidomimetic thereof, can act, for example, by competing with the corresponding normal component of the complex and specifically associating with the other components of the complex, but can lack a kinase site such that the dominant negative form of the kinase is not phosphorylated and, therefore, does not propagate signal transduction.

A method of the invention can be performed in vitro using substantially purified components, including JNK and MKK polypeptides and, where appropriate, a scaffold protein and/or a MAP3K polypeptide; or using cell extracts that contain JNK, MKK, scaffold protein, and/or MAP3K polypeptides; or can be performed in a cell. An advantage of using an in vitro system is that it is unnecessary to isolate or maintain living cells, thus reducing the costs of the assays. In addition, in vitro assays can be conveniently standardized and, therefore, the reagents for performing the assays can be provided in the form of a kit, which can include, for example, standardized amounts of a JNK, MKK, scaffold protein, and/or MAP3K polypeptide, and reagents such as buffers, thus providing precisely defined conditions for each assay of a plurality of assay and simplifying comparison of results in assays performed serially or in parallel. An advantage of using a cell based system is that the test agent, to be effective, must traverse the cell membrane and, therefore, a positive result indicates that the agent can be useful, for example, for in vivo purposes. In addition, a cell based assay allows for the identification of an agent that alters MAP3K/MKK or JNK/MKK formation or activity by affecting the intracellular translocation of a MAP3K or MKK polypeptide, or a complex comprising a MAP3K or MKK polypeptide, An alteration that affects intracellular translocation of one or more components of a protein complex can be detected, for example, by confocal microscopy (see Example 1).

Where a method of the invention is performed in vitro, the reaction mixture contains appropriate buffers, salts, metal ions and, where appropriate, reagents that may be required for detecting an affect due to the test agent, for example, a phosphate transfer group such as provided by ATP or GTP, or a substrate such as c-Jun, and the like. Where the method is performed in a cell, the cell is selected such that it expresses a JNK, MKK, JIP-3, and/or MAP3K. Either or all of the JNK, MKK, scaffold protein, and MAP3K polypeptides can be endogenous to the cell, i.e., expressed from genes normally present in the cells, or the polypeptides can be heterologous polypeptides, which can be expressed from a polynucleotide that is introduced into the cell under conditions such that the polypeptide can be expressed. Polynucleotides encoding JNK polypeptides, including JNK1, JNK2, and JNK3; MKK polypeptides such as MKK4 and MKK7; scaffold proteins such as JIP polypeptides (e.g., JIP-3), and MAP3K polypeptides such as MEKK1, MEKK2, and TAK1 are well known in the art and disclosed, for example, on the world wide web, at the URL “ncbi.nlm.nih.gov” (for example, human JNK1, GenBank Acc. No. L26318; human JNK2, GenBank Acc. No. L30951; human JNK3A1, GenBank Acc. No. U3482; human MKK4, GenBank Acc. No. NM_{—}003010; human MKK7, GenBank Acc. Nos. AF013558; murine MEKK1, GenBank Acc. No. 117340; human MEKK2, GenBank Acc. No. AF111105; human TAK1, GenBank Acc. No. NM_{—}145332; and murine JIP-3, GenBank Acc. No. AF109771, each of which is incorporated herein by reference; see, also, Lin et al., supra, 1995; Tournier et al., supra, 1997; Moriguchi et al., supra, 1997, including GenBank Acc. Nos. cited therein).

A polynucleotide encoding a JNK, MKK, scaffold protein, or MAP3K polypeptide, which can be contained in a vector, particularly an expression vector, can be introduced into a cell by any of a variety of methods known in the art (see, for example, Sambrook et al., “Molecular Cloning: A laboratory manual” (Cold Spring Harbor Laboratory Press 1989); Ausubel et al., “Current Protocols in Molecular Biology”, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

For expression in a cell, the polynucleotide encoding the JNK, MKK, scaffold protein, and/or MAP3K generally is operatively linked to one or more transcriptional regulatory elements, including, for example, one or more promoters, which comprise a transcription start site; enhancers or silencers, which increase or decrease, respectively, the level of transcription of an expressible nucleotide sequence; or terminators, which comprise a transcription stop site. Promoters and enhancers, which can be used to drive transcription of the polynucleotide can be constitutive (e.g., a viral promoter such as a cytomegalovirus promoter or an SV40 promoter), inducible (e.g., a metallothionein promoter), repressible, or tissue specific, as desired. As used herein, the term “operatively linked” means that a regulatory element is positioned with respect to an expressible polynucleotide sequence such that the element can effect its regulatory activity. A transcriptional regulatory element having enhancer activity, for example, can be located at some distance, including adjacent to or up to thousands of nucleotides away from, and upstream or downstream from a promoter and a nucleotide sequence to be transcribed, and still exert a detectable enhancing effect on the level of expression of an encoded reporter molecule. Transcriptional regulatory element, including eukaryotic and prokaryotic promoters, terminators, enhancers, and silencers, are well known in the art and can be chemically synthesized, obtained from naturally occurring nucleic acid molecules, or purchased from commercial sources.

Where a screening assay of the invention is performed using a cell based system, the cell generally is a vertebrate cell such as a mammalian cell, and particularly is a human cell such as a synoviocyte, which can be a synoviocytes obtained from a subject suffering from an arthritis such as rheumatoid arthritis, osteoarthritis, or other inflammatory arthritis. Thus, the cell can be a cell in culture, including a cell of a cell line that has been adapted to passage is tissue culture, or a cell that is removed from a subject and contacted ex vivo. Furthermore, panels of cells can be made available for use in a method of the invention, including, for example, cell lines derived from synoviocytes from a healthy individual, synoviocytes from a subject with various types of arthritis, synoviocytes from a subject with a particular type of arthritis such as OA or RA, synoviocytes from a subject with an non-arthritis autoimmune disorder, and the like. Accordingly, the present invention provides agents that are identified according to a method of the invention, as well as compositions useful as medicaments for ameliorating a disorder associated with abnormally elevated JNK signalsome activity in cells of a subject, particularly an inflammatory arthritis.

The present invention provides a method of ameliorating a disorder such as an arthritis in a subject, wherein the disorder is associated with cells exhibiting abnormal JNK signalsome mediated signal transduction. Rheumatoid arthritis (RA) and osteoarthritis (OA) are provided as examples of such disorders, wherein JNK signalsome mediated signal transduction in RA or OA synoviocytes is abnormally elevated as compared to corresponding synoviocytes from a healthy individual. In view of the disclosed upstream components of the JNK signal transduction pathway, including MAP3K proteins such as MEKK1, MEKK2 and TAK1, of MKK proteins such as MKK4 and MKK7, of scaffold proteins such as JIP-1 and JIP-3, and their roles in JNK signalsome mediated signal transduction, it will be recognized that additional disorders similarly can be identified and treated according to the present methods.

As used herein, the term “ameliorate” means that signs or symptoms associated with the disorder associated with cells exhibiting abnormal JNK signalsome mediated signal transduction are lessened. The signs or symptoms to be monitored will be characteristic of the particular disorder and will be well known to skilled clinician, as will the methods for monitoring the signs and conditions. For example, where the disorder is rheumatoid arthritis, the clinician will know that increased joint mobility or dexterity exhibited by the subject; or decreased inflammatory response as evidenced by lower cytokine levels or fewer inflammatory cells; or reduced level or decreased rate of joint and/or cartilage destruction as evidenced by an imaging method, are indicative of a method of the invention ameliorating the arthritis. Similarly, where the disorder is osteoarthritis, the clinician will know that a decrease in joint and/or cartilage damage is indicative of ameliorating the arthritis.

As used herein, the term “abnormal”, when used in reference to JNK signalsome mediated signal transduction, means that the level of signal transduction in a cell being examined is different from the level that is generally characteristic for the particular cell type, i.e., is not normal. As such, the level can JNK signalsome mediated signal transduction can be increased above or decreased below normal, or can be stimulated for a longer or shorter period of time than normal. The term “normal” is used herein to refer to a mean value, which can include a range of values comprising the mean bounded by one or a few standard deviations, that is generally found in cells of a healthy population of individuals, which can be a random population or specifically biased population. A normal value for JNK signalsome mediated signal transduction in particular cell types can be determined using routine statistical sampling methods. For example, cells such as synoviocytes can be obtained from a population of healthy individuals and tested for the level or activity of JNK signalsome formation or activity and, therefore, JNK mediated signal transduction, for example, by examining the level of collagenase gene expression that occurs under basal or induced conditions, and the mean and standard deviation of the measured levels or activities can be determined. If desired, the population of individuals examined can be a randomly selected population, or can be a biased population, in which individuals within a specified age range; or all males or all females, or a combination of males and females; or the like, are selected. By comparing, for example, the level of JNK mediated signal transduction in a test cell with a corresponding cell known to be normal, or with a value known to be normal, a determination can be made as to whether the test cell exhibits an abnormal level of JNK signalsome mediated signal transduction and, therefore, is amenable to treatment according to a method of the invention.

A method of ameliorating a disorder such as an inflammatory arthritis, which is associated with synoviocytes exhibiting abnormally elevated JNK signalsome mediated signal transduction, can be performed, for example, by contacting the affected cells of the subject with an agent that reduces or inhibits MAP3K/MKK and/or JNK/MKK protein complex formation or activity, thereby reducing or inhibiting JNK signalsome mediated signal transduction in the cells and ameliorating the disorder. The cells of the subject can be contacted with the agent by administering the agent to the subject, for example, systemically such that the agent circulates to the cells, to directly to one or more sites of an arthritis (and the cells) in the subject. Cells of the subject also can be contacted with the agent ex vivo, then the cells that have been contacted with the agent can be administered back into the subject to the subject, for example, to a site of the arthritis in the subject.

For administration to a living subject, including a human or other subject, the agent generally is formulated with a pharmaceutically acceptable carrier to provide a composition suitable for administration the subject. The form of the composition will depend, in part, on the route by which the composition is to be administered. Generally, the composition will be formulated such that the agent is in a solution or a suspension, such a form be suitable for administration by injection, infusion, or the like, or for aerosolization for administration by inhalation. However, the composition also can be formulated as a cream, foam, jelly, lotion, ointment, gel, or the like. Alternatively the agent can be formulated in an orally available

A pharmaceutically acceptable carrier useful for formulating a composition for use in a method of the invention can be aqueous or non-aqueous, for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the agent, for example, carbohydrates, such as glucose, sucrose or dextrans, antioxidants, such as ascorbic acid or glutathione, chelating agents, low molecular weight proteins or other stabilizers or excipients.

The pharmaceutical composition also can comprise an admixture with an organic or inorganic carrier or excipient, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary stabilizing, thickening or coloring agents can be used, for example a stabilizing dry agent such as triulose.

Where a polynucleotide agent is used, it can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton Fla. 1984); Fraley et al., Trends Biochem. Sci., 6:77, 1981). Liposomes, for example, which consist of phospholipids or other lipids, are nontoxic, physiologically acceptable and metabolizable carriers that are relatively simple to make and administer. “Stealth” liposomes (see U.S. Pat. Nos. 5,882,679; 5,395,619; and 5,225,212) are an example of such encapsulating materials particularly useful for preparing a pharmaceutical composition.

A polynucleotide agent can be constructed of naturally occurring nucleotides, or nucleotide analogs, including non-naturally occurring synthetic nucleotides or modified naturally occurring nucleotides, can be used to construct the polynucleotide agent. Such nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference). Similarly, while the covalent bond linking the nucleotides of a polynucleotide agent generally is a phosphodiester bond, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994); Ecker and Crooke, BioTechnology 13:351-360, 1995, each of which is incorporated herein by reference). The incorporation of non-naturally occurring nucleotide analogs or bonds linking the nucleotides or analogs can be particularly useful where the polynucleotide agent is to be exposed to an environment that can contain a nucleolytic activity, including, for example, a tissue culture medium or upon administration to a living subject such as into a joint of a subject with arthritis, since the modified nucleic acid molecules can be less susceptible to degradation.

Similarly, a peptide agent can contain naturally occurring amino acids and peptide bonds, or can be a modified peptide containing, for example, one or more D-amino acids in place of a corresponding L-amino acid; or one or more amino acid analogs, for example, an amino acid that has been derivatized or otherwise modified at its reactive side chain, or the peptide can be modified at its amino terminus or the carboxy terminus or both. Such peptides can have improved stability to a protease, an oxidizing agent or other reactive material the peptide may encounter in a biological environment, and, therefore, can be particularly useful in performing a method of the invention. Of course, the peptides can be modified to have decreased stability in a biological environment such that the period of time the peptide is active in the environment is reduced.

The amount of the particular agent contained in a composition can be varied, depending on the type of composition, such that the amount present is sufficient to increase or decrease the JNK signalsome mediated signal transduction, as appropriate, thereby ameliorating the disorder. In general, an amount of an agent sufficient to provide a therapeutic benefit will be determined using routine clinical methods, including Phase I, II and III clinical trials.

The present invention also provides an isolated protein complex, including, a JNK/MKK4 complex, a JNK/MKK7 complex, a JNK/MKK4/MKK7 signalsome, a scaffold protein/JNK signalsome complex (e.g., a JIP-3/JNK/MKK4/MKK7 complex), an MEKK1/MKK4, MEKK2/MKK4, or TAK1/MKK4 complex, and an MEKK1/MKK7, MEKK2/MKK7 or TAK1/MKK7 complex. In addition, the invention provides an antibody that specifically binds such a protein complex, provided the antibody does not substantially bind to any of an isolated JNK polypeptide, an isolated JIP polypeptide, an isolated MKK4 polypeptide, an isolated MKK7 polypeptide, or an isolated MEKK2 polypeptide. As disclosed herein, such protein complexes and antibodies are useful for practicing the methods of the invention. Accordingly, the present invention further provides kits, which contain one or more of the above protein complexes or antibodies, or combinations thereof.

The following examples are intended to illustrate but not limit the invention.

### EXAMPLE 1

### JNK Signalsome Regulates JNK Mediated Signal Tranduction in Arthritis Synoviocytes

This example demonstrates that protein complexes, including a JNK/MKK4 and a JNK/MKK7 complex, and JNK signalsome comprising JNK, MKK4 and MKK7 specifically form in RA and OA synoviocytes to activate JNK mediated signal transduction.

Synovial tissue (ST) samples were obtained from patients with osteoarthritis (OA) and rheumatoid arthritis (RA) at the time of joint replacement as described by Alvaro-Garcia (J. Clin. Invest. 86:1790-1798, 1990, which is incorporated herein by reference). The diagnosis of RA conformed to the 1987 revised American college of rheumatology criteria (Arnett et al., Arthritis Rheum. 31:315-324, 1988). The samples were either processed for cell culture or snap frozen and stored at −80°C.

To obtain fibroblasts-like synoviocytes (FLS) synovial tissues were minced, incubated with 1 mg/ml collagenase in serum free DMEM for 2 hr at 37° C., filtered through a nylon mesh, extensively washed, and cultured in DMEM supplemented with 10% FCS (endotoxin content <0.006 ng/ml), penicillin, streptomycin, and L-glutamine in a humidified 5% CO₂ atmosphere. After overnight culture, nonadherent cells were removed, and adherent cells were trypsinized, split at a 1:3 ratio, and cultured in medium. Synoviocytes were used from passages 3 through 9 in these experiments, during which time they comprised a homogenous population of FLS (<1% CD11b, <1% phagocytic, and <1% FcγRII positive.

Affinity purified rabbit polyclonal anti-MKK4 antibodies, goat polyclonal anti-MKK7 antibodies, and anti-goat-HRP (horse radish peroxidase) conjugated antibodies were purchased from Santa Cruz Biotechnology (Santa Cruz Calif.). Mouse monoclonal anti-phospho-JNK antibodies, rabbit polyclonal anti-phospho-MKK4 antibodies, anti-phospho-MKK7 antibodies, and anti-rabbit-HRP-conjugated Ab were obtained from New England BioLabs, Inc. (Beverly Mass.). Interleukin-1β (IL-1) was purchased from R&D Systems (Minneapolis Minn.).

For western blot analysis, cells were cultured in DMEM with 10% FCS in 100 mm dishes at 80% confluency. Forty-eight hr before stimulation, cells were synchronized in DMEM by culturing in 0.1% FCS, then FLS were stimulated with medium or IL-1 (2 ng/ml) for 15 min, or different time points as indicated. Cells were washed with phosphate buffered saline (PBS), and protein was extracted using RIPA buffer (50 mM Tris-HCl (pH 7.5), 150 mM NaCl, 1% Nonidet P-40 detergent, 0.5% sodium deoxycholate and 0.1% SDS). Frozen ST was pulverized, and tissue protein was extracted in the same manner. The protein concentrations were determined using the DC Protein assay kit (BioRad). Samples containing 40 μg of protein from cultured FLS or 300 μg of protein from ST were fractionated by 10% SDS-PAGE and transferred to nitrocellulose membrane at 140 mA in 25 mM Tris-HCl, pH 8.3, 192 mM glycine and 50% methanol.

Western blot analysis was performed with antibodies specific for MKK4, MKK7, phospho-MKK4 or phospho-MKK7 according to the manufacturer's instructions. Briefly, filters were blocked with Tris buffered saline (TBS) plus 0.1% Tween-20 detergent and 5% dry milk for 1 hr at room temperature (RT), followed by incubation with appropriate antibody at 4° C. overnight. The membrane was washed three times and incubated with HRP conjugated secondary antibody for 1 hr at RT. Proteins were visualized by chemiluminescence using Kodak X-AR film (Eastman Kodak Co.; Rochester N.Y.). The density of target bands was analyzed using National Institute of Health Image software (version 1.61; NIH; Bethesda Md.). Results are expressed as arbitrary densitometry units (AU).

Immunohistochemistry was performed as described by Tak et al. (Arthritis Rheum. 44:1897-1907,2001, which is incorporated herein by reference). Four μM cryosections of ST cut from RA and OA patients were obtained, fixed in cold acetone for 10 min, and incubated with anti-MKK4, anti-MKK7, anti-phospho-MKK4 or anti-phospho-MMK7 antibodies overnight at 4° C.; isotype matched antibodies served as negative controls. Endogenous peroxidase was depleted with 0.3% hydrogen peroxide, then sections were stained with biotinylated secondary antibody (Vector; Burlingame Calif.). The signal was developed using diaminobenzidine or AEC reagent, and the sections were counterstained with hematoxylin. The synovial tissue in the lining and sublining region were evaluated for the presence MKK4, MKK7 or P-MKK4 or P-MKK7 using the semiquantitaive scoring system: 0, no staining; 1+, rare positive staining or trace staining (1-5%); 2+, scattered clusters of positive cells (6-15%); 3+, moderate staining in specific region (16-50%); and 4+, extensive staining through out the region (51-100%; see Tak et al., Arthritis Rheum. 42:948-953, 1999).

For immunofluorescence, FLS were cultured in eight chamber poly L-lysine coated glass slides (10,000 cells/well; Nunc; Naperville Ill.). After 16 hr, medium was replaced with 0.1% FCS for 48 hr, then the cells were stimulated with 0.1 % FCS/DMEM or IL-1 (2 ng/ml). Cells were washed twice with PBS, fixed with 3.7% formaldehyde for 10 min, and permeabilized with 0.1 % saponin. The cells were blocked with 20 mg/ml BSA for 1 hr at RT, then incubated with primary antibody to anti-MKK4, anti-MKK7, or anti-JNK antibody at 1:50 or anti-phospho-JNK antibody at 1:100 in 20 mg/ml BSA overnight at 4° C. Cells were then washed with PBS and incubated with anti-rabbit FITC conjugate (Pharmingen; San Diego Calif.), anti-goat Texas red conjugate, or anti-mouse CY5 conjugate (1:1000; Molecular Probes, Inc.; Eugene Oreg.). After extensive washing, cells were counter stained with Hoechst stain at 10 μg/ml, mounted with gelvitol mounting reagent, air dried, and analyzed using laser confocal microscopy. Images were captured with a DeltaVision deconvolution microscope system (Applied Precision, Inc.; Issaquah Wash.). Between 30 and 80 optical sections spaced by 0.1-0.3 μm were examined. Exposure times were set such that the camera response was in the linear range for each fluorophore. The data sets were deconvoluted and analyzed using SoftWorx software (Applied Precision, Inc) on a Silicon Graphics Octane workstation. Maximal projection volume views or single optical sections were obtained.

For in vitro kinase assays, cells were cultured in DMEM with 10% FCS in 100 mm dishes to 80% confluence. Forty-eight hr before stimulation, cells were synchronized in DMEM with 0.1% FCS, then the cells were incubated with 0.1 FCS/DMEM or IL-1 (2 ng/ml) for 15 min. Kinase assays were performed as described by Hibi et al. (Genes Devel. 7:2149-2160, 1993, which is incorporated herein by reference), except using modifications as follows. Cells were washed with cold PBS two times, scraped directly into lysis buffer (50 mM HEPES, pH 7.4, 150 mM NaCl, 1.5 mM MgCl₂, 1 mM EGTA, 10% glycerol, 1% Triton X-100, 20 mM β-glycerophosphate, 10 mM sodium fluoride, 1 mM Na₃VO₄, 10 μg/ml aprotinin, 1 μM pepstatin A, 1 mM PMSF), and allowed to stand for 30 min.

The homogenate was then centrifuged for 10 min at 14,000×g, and the supernatant was retained for immunoprecipitation. Samples containing 500 μg of protein were incubated with a specific anti-phospho-MKK4 or anti-phospho-MKK7 antibodies, then the complex was incubated for 4 hr at 4° C. Forty μl of a 50% protein A-Sepharose 4B-Cl gel slurry in lysis buffer was added, then the mixture was incubated for overnight on a rotation wheel. After centrifugation for 5 min at 1000×g, immuncomplexes were washed three times with lysis buffer and once with kinase buffer (25 mM HEPES, pH 7.4, 25 mM MgCl₂, 20 mM β-glycerophosphate, 0.1 mM Na₃VO₄, 2 mM dithiothreitol). The kinase reaction was started by adding the 30 μl of kinase buffer with GST-c-Jun polypeptide as substrate at 2 μg per reaction (25 mM HEPES, pH 7.4, 25 mM MgCl₂, 20 mM β-glycerophosphate, 0.1 mM Na₃VO₄, 2 mM dithiothreitol, 20 μM ATP, 2 μCi γ³²P-ATP for 30 min at 37° C. Samples were heated for 5 min at 95° C., separated using 10% SDS-PAGE, and visualized by autoradiography.

Statistical analysis was performed by unpaired Student's t-test, unless indicated otherwise. A comparison was considered statistically significant if p<0.05.

Expression and Activation of MKK4 and MKK7 in Synovial Tissues

To identify the key upstream activators of JNK in RA, immunohistochemistry studies were performed to determine if MKK4 and MKK7 are expressed in synovial tissue. MKK4 and MKK7 were primarily detected in the synovial intimal lining, although positive cells were also identified in the subliming region. No differences were noted between RA and OA utilizing a semiquantitative scoring system. Serial sections stained with isotype matched control antibodies were negative. Using anti-phospho-MKK antibodies, prominent phospho-MKK4 and phospho-MKK7 expression was observed in the intimal lining and endothelial cells of RA synovium and small amounts of phospho-MKK4 and phospho-MKK7 were detected in OA synovial tissue (n=6 RA and 5 OA). Serial sections stained with isotype matched control antibodies were negative.

Western blot analysis was performed on lysates of RA and OA synovium using specific anti-MKK4 and anti-MKK7 antibodies. Abundant MKK4 and MKK7 were detected in the extracts, and no differences were observed between OA and RA tissues. Western blot analysis also was performed using antibodies specific for the phosphorylated MKKs. Phospho-MKK4 and Phospho-MKK7 were found to be expressed in both RA and OA. When normalized for protein content, there was significantly more phosphorylated MKK4 and MKK7 in RA compared with OA synovium (n=3 RA and 3 OA lines).

Expression and Phosphorylation of MKK4 and MKK7 in FLS

Previous studies demonstrated that JNK is a key regulator of MMP expression in fibroblast-like synoviocytes in the synovial intimal lining. Based on the observation that MKK4 and MKK7 were activated in the same region, attention was focussed on the regulation of these MKKs in cultured FLS isolated from synovial tissue. Initial studies using western blot analysis showed that MKK4 and MKK7 are constitutively expressed in RA and OA FLS. To explore the time course of MKK4 and MKK7 activation by IL-1, FLS were synchronized in 0.1% FCS for 48 hr, then stimulated with 2 ng/ml of IL-1 for 15 min to 60 min (n=2 RA and 2 OA lines). Western blot analysis revealed that both MKK4 and MKK7 were rapidly phosphorylated, reaching a maximum at 15 min, with a subsequent decrease after 60 min. No difference was observed between OA and RA FLS as to the timing or extent of phosphorylation.

Components of JNK Signaling Complex in FLS

JNK activation requires phosphorylation of both Tyr and Thr within the Thr-Pro-Tyr motif by a combination of MKK7 and MKK4 (Fleming et al., supra, 2000). In addition, MKK7, but not MKK4, forms a signaling complex with JNK in murine embryonic cells (Tournier et al., supra, 2001). Immunoprecipitation experiments were performed to determine the components of the JNK signaling complex in FLS (n=3 RA and 3 OA lines). Cells were stimulated with medium or IL-1, then the lysates were immunoprecipitated with anti-MKK4 or anti-MKK7 antibody. JNK and MKK7 were detected in the MKK4 immunoprecipitate (anti-MKK4 antibody) and, similarly, the JNK and MKK4 were detected in the MKK7 immunoprecipitate. These results indicate that MKK4, MKK7 and JNK specifically associate to form a protein complex in FLS.

Since the putative JNK scaffold protein, JNK interacting protein (JIP-1), can form complexes with JNK and MKK7 (Tournier et al., supra, 2001), co-precipitation experiments were performed using anti-JIP-1 antibody. As in murine embryonic cells, MKK7, but not MKK4, immunoprecipitates contained JIP-1. In contrast, JIP-3 was detected in MKK4 and MKK7 immunoprecipitates. These results indicate that at least two JNK signal complexes are present in FLS—a MKK7/JNK/JIP-1 complex and a MKK7/MKK4/JNK/JIP-3 complex.

Localization of MKK4, MKK7 and JNK in FLS

MAPK family members can be localized to either the cytoplasm or the nucleus, depending on the specific kinase and the type of stimulation. To localize JNK and its upstream activators in FLS, cells were analyzed by immunofluorescence and confocal microscopy. Cultured FLS were stimulated with medium or 2 ng/ml of IL-1 for 15 to 60 min, then stained with antibodies specific for MKK4, MKK7 or JNK. In resting cells, all three kinases were located primarily in the cytoplasm of resting cells. After 60 min of IL-1 stimulation, all three kinases were detected in the cytoplasm, and a portion had migrated to the perinuclear region or the nucleus. Additional studies using anti-phospho-JNK antibody showed increased cytoplasmic, then nuclear localization after IL-1 stimulation.

Functional Activation of the JNK Signalsome

To determine whether the MKK4/MKK7/JNK signalsome is functionally active, FLS were stimulated with medium or IL-1 and the complex was immunoprecipitated with anti-MKK4 or anti-MKK7 antibody. In vitro kinase assays were performed using GST-c-Jun as the substrate because it is phosphorylated by JNK but not the MKKs (n=4 RA and 3 OA lines). Both the MKK4 and MKK7 immunoprecipitates formed functionally active complexes with JNK, as indicated by their ability to phosphorylate GST-c-Jun following IL-1 stimulation of FLS (n=3). To confirm that the phosphorylation of GST-c-Jun observed following incubation with these complexes was due to JNK, FLS were stimulated by medium or IL-1 for 15 min and the specific JNK inhibitor SP600125 (2 μM; see Example 2) was added during the kinase reaction. The ability of the MKK4 or MMK7 immunoprecipitates to phosphorylate GST-c-Jun was inhibited SP600125 (n=1 RA and 1 OA line). These results demonstrate that both MKK4 and MKK7 form an active complex with JNK in FLS and activate JNK.

In summary, phospho-MKK4 and phospho-MKK7 expression was detected in RA synovium and, to a lesser extent, in OA synovium, especially in the intimal lining. The phosphoproteins were present in complexes with JNK, and were localized in FLS cells in the regions where AP-1 proteins and MMPs are expressed. These results using FLS contrast with the results obtained in murine embryonic fibroblasts, where only MKK7 (but not MKK4) was activated by cytokines (Tournier et al., supra, 2001), indicating that the regulation of MKK4 activation varies with cell type and culture conditions.

These results indicate that MKK4, MKK7, and JNK can form multiple signaling complexes in FLS, which can facilitate phosphorylation of JNK by distinct stimuli and permit cells to integrate the extracellular stresses and generate the appropriate physiologic response. Because MKK4 and MKK7 have a greater than additive effect in their ability to activate JNK, the JNK signalsome, JNK/MKK4/MKK7, can have a central role in cytokine mediated MAPK activation and expression of AP-1 regulated genes such as those encoding collagenases and stromelysin, which can contribute to joint destruction in arthritis patients. As such, agents that inhibit the activity of the individual components of these complexes, the nuclear migration of the components or complexes, or the ability of the kinases to specifically associate into the complexes can provide a therapeutic benefit to arthritis patients.

### EXAMPLE 2

### Specific Inhibitor of JNK Reduces Collagenase Gene Expression in Synoviocytes and Joint Damage In Arthritis Animal Model

SP600125 (anthra {1,9-cd} pyrazol-6(2H)-one; Signal Pharmaceuticals, Inc.; San Diego Calif.) is a selective inhibitor of JNK, having at least 100-fold selectivity for JNK when examined against a panel of more than forty enzymes (see Table 1; see, also, Han et al., supra, 2001). The effect of SP600125 and MAP kinase inhibitors on collagenase gene expression was examined to determine whether JNK is involved in metalloproteinase gene activation in FLS. FLS were stimulated with medium, IL-1 (2 ng/ml), or IL-1+SP600125 (0-50 μM) for 18 hr. RNA was extracted and northern blot analysis was performed to determine the collagenase mRNA accumulation (Han et al., supra, 2001).

TABLE 1
SPECIFICITY OF SP600125 JNK INHIBITOR

| Enzyme | IC50 (μM) | Enzyme | IC50 (μM) |
|---|---|---|---|
| JNK1 | 0.04 | Phospholipase A2 | >10 |
| JNK2 | 0.04 | Adenylate cyclase | >10 |
| JNK3 | 0.09 | Guanylate cyclase | >10 |
| ERK2 | >10 | ATPase (Na/K) | >10 |
| p38-β | >10 | Protein kinase C | >10 |
| I kappaB kinase-2 | >10 | HIV-1 protease | >10 |
| Acetylcholine | >10 | Monoamine oxidase | >10 |
| esterase | | | |
| cycloxygenase-2 | >10 | Tyrosine hydrolase | >10 |
| 5′-lipoxygenase | >10 | Elastase | >10 |
| PDE I, III, IV, V | >10 | Cathepsin B | >10 |
| INOS | >10 | Cathepsin G | >10 |

IL-1 induced collagenase expression and the increase was inhibited by SP600125. Like many small molecule inhibitors, the concentration required in cells is higher than the IC50 for the isolated enzyme. The p38 inhibitor SB203580 had no effect on collagenase expression at concentrations that completely blocked p38 while the MEK/ERK inhibitor PD98059 decreased expression at the highest concentration. These results indicate that the JNK signal transduction pathway is involved in IL-1 induced collagenase regulation.

The effect of JNK inhibition by SP600125 on matrix destruction was examined in vivo in an animal model of arthritis. Rats were immunized on day 0 with complete Freund's adjuvant and treated with SP600125 (30 mg/kg/d subcutaneous (sc)) or vehicle beginning on day 8 (n=8 animals/group). Measurement of paw volumes revealed that the JNK inhibitor modestly decreased swelling in the treated rats. Furthermore, radiographic analysis at the conclusion of the study demonstrated a significant decrease in bone and cartilage damage in the treated animals (see Han et al., supra, 2001). Joint destruction was quantified using a scoring system that measures calcaneal involvement, erosions, demineralization and heterotopic bone formation (maximum score=6).

To determine the cause for the decreased radiographic joint destruction in adjuvant arthritis, rats were immunized on day 0 with complete Freund's adjuvant and treated with SP600125 (30 mg/kg/d sc) or vehicle beginning on day 8 (n=4 animals/group). On day 20, animals were sacrificed and synovial RNA extracts were prepared from the ankle joints. Northern blot analysis revealed that MMP13 (collagenase 3) expression was decreased in the rats treated with the JNK inhibitor as compared to those treated with vehicle (P<0.01 for G3PDH normalized values).

### EXAMPLE 3

### MEKK2 Associates with MKK4 and MKK7 in Rheumatoid Arthritis Synoviocytes

This example demonstrates that the MAP kinase kinase kinase (MAP3K), MEKK2, form a functional complex with MKK4 and MKK7 in RA FLS.

Expression of the MAP3Ks, MEKK1, MEKK2, MEKK3, MEKK4, ASK-1, TAK1 and MLK3, was examined in cultured normal FLS, RA FLS, and OA FLS. mRNA and protein levels were assessed by northern blot analysis and western blot analysis, respectively. Similar studies were performed on RA and OA synovial tissue. To determine the composition of MAP3K/MAP kinase kinase complexes, serum starved FLS were stimulated with IL-1 (2 ng/ml) or with normal medium for 15 min, then immunoprecipitated using anti-MEKK1, anti-MEKK2, or anti-TAK1 antibodies, and probed with either anti-MKK4 or anti-MKK7 antibodies (Santa Cruz Biotechnology). Kinase activity of MEKK1, MEKK2, and TAK-1 was measured in vitro using GST-MKK4, GST-MKK7, or GST-c-Jun as substrates.

Expression of all of the MAP3K mRNA molecules was detected in RA, OA and normal FLS, and there was no difference in expression between RA, OA, and normal FLS. Western blot analysis of FLS lysates and mRNA studies indicated that MEKK1, MEKK2 and TAK1 protein were the most abundant. MEKK3, ASK-1 and MLK3 proteins were present at low levels; MEKK4 was not detected. Similar results were obtained with the RA and OA synovial tissues; MEKK1, MEKK-2 and TAK1 mRNA and protein were most abundant. Immunoprecipitation assays revealed that MEKK2 co-precipitated with MKK4 and MKK7, and kinase assays showed that MEKK2 phosphorylates MKK4 and MKK7.

These results demonstrate that multiple MAP3Ks, particularly MEKK2, MEKK1 and TAK1, are expressed in FLS and arthritic synovium, and that MEKK2 forms a functional complex with MKK4 and MKK7, and can phosphorylate these MAP kinase kinases. As such, MEKK2 is upstream of MKK4 and MKK7 in the JNK signal transduction pathway in FLS cells, including in RA and OA synoviocytes, and in arthritic tissues.

Although the invention has been described with reference to the above example, it will be understood that modifications and variations are encompassed within the spirit and scope of the invention. Accordingly, the invention is limited only by the following claims.

What is claimed is:

1. A method of identifying an agent that modulates c-Jun N-terminal kinase (JNK) mediated signal transduction in a synoviocyte, the method comprising:
a) contacting the synoviocyte, or an extract of a synoviocyte with a test agent; and
b) detecting a change in the formation or activity of at least one protein complex selected from
i) a JNK/MAP kinase kinase (MKK) protein complex, wherein the MKK is MKK4 or MKK7;
ii) a JNK/MKK4/MKK7 protein complex,
iii) a JNK interacting protein (JIP)/JNK/MKK4/MKK7 protein complex, wherein the JIP is JIP-1 or JIP-3,
iv) a MAP kinase kinase kinase (MAP3K)/MKK4 protein complex, wherein the MAP3K is MEKK1, MEKK2, or TAK1, or
vi) a MAP3K/MKK7 protein complex, wherein the MAP3K is MEKK1, MEKK2, or TAK1,
in the presence of the test agent as compared to the formation or activity of the protein complex in the absence of the test agent, thereby detecting an agent that modulates JNK mediated signal transduction in a synoviocyte.

2. The method of claim 1, wherein the synoviocyte is a synoviocyte from a subject with arthritis.

3. The method of claim 1, wherein the synoviocyte is a synoviocyte from a subject with rheumatoid arthritis or osteoarthritis.

4. The method of claim 1, further comprising contacting the synoviocyte with a cytokine.

5. The method of claim 4, wherein the cytokine is interleukin-1.

6. The method of claim 4, wherein contacting the synoviocyte with the cytokine precedes contacting the synoviocyte with the test agent.

7. The method of claim 4, wherein contacting the synoviocyte the cytokine is substantially simultaneous with contacting the synoviocyte with the test agent.

8. The method of claim 1, wherein the extract is an extract of a synoviocyte contacted with a cytokine.

9. The method of claim 1, wherein detecting the change in formation of activity of the protein complex in the presence of the test agent comprises detecting a reduced level of formation or activity protein complex.

10. The method of claim 1, wherein detecting the change in formation of activity of the protein complex in the presence of the test agent comprises detecting disruption of the protein complex.

11. An isolated agent that modulates JNK signal transduction activity in a synoviocyte, wherein the agent is identified by the method of claim 1.

12. A method of identifying an agent that modulates c-Jun N-terminal kinase (JNK) mediated signal transduction in a synoviocyte, the method comprising:
a) contacting
1)
i) a c-Jun N-terminal kinase (JNK),
ii) a MAP kinase kinase (MKK), wherein the MKK is MKK4, MKK7, or both MKK4 and MKK7, and, optionally,
iii) a scaffold protein, under conditions suitable for JNK/MKK protein complex formation or activity, and
2) a test agent; and
b) detecting a change in JNK/MKK protein complex formation or activity in the presence of the test agent as compared to the JNK/MKK protein complex formation or activity in the absence of the test agent, thereby detecting an agent that modulates JNK mediated signal transduction in a synoviocyte.

13. The method of claim 12, wherein the JNK/MKK protein complex comprises a JNK/MKK4 complex, a JNK/MKK7 complex, or a JNK/MKK4/MKK7 complex.

14. The method of claim 12, wherein the scaffold protein is a JNK interacting protein (JIP).

15. The method of claim 14, wherein the JIP is JIP-1 or JIP-3.

16. The method of claim 15, wherein the JNK/MKK protein complex comprises a JNK/MKK4/MKK7/JIP-3 complex.

17. The method of claim 12, comprising contacting the JNK and MKK, wherein a JNK/MKK protein complex forms, and thereafter contacting the JNK and MKK with the test agent.

18. The method of claim 12, further comprising contacting the JNK and MKK with a MAP kinase kinase kinase (MAP3K), under conditions suitable for MAP3K/MKK protein complex formation or activity.

19. The method of claim 18, wherein the MAP3K is MEKK1, MEKK2, or TAK1.

20. The method of claim 12, wherein the synoviocyte is a cytokine stimulated synoviocyte.

21. The method of claim 12, wherein the synoviocyte is an inflammatory arthritis synoviocyte.

22. The method of claim 12, wherein detecting a change in JNK/MKK protein complex formation or activity in the presence of the test agent comprises detecting a reduced level of the JNK/MKK protein complex formation or activity.

23. The method of claim 12, wherein detecting a change in JNK/MKK protein complex formation or activity in the presence of the test agent comprises detecting disruption of the JNK/MKK protein complex.

24. The method of claim 12, wherein detecting a change in JNK/MKK protein complex formation or activity in the presence of the test agent comprises detecting an increased level in the JNK/MKK protein complex formation or activity.

25. The method of claim 12, wherein detecting a change in JNK/MKK protein complex formation or activity in the presence of the test agent comprises detecting stabilization of the JNK/MKK protein complex.

26. The method of claim 12, wherein detecting a change in JNK/MKK protein complex formation or activity in the presence of the test agent comprises detecting a reduced level of MKK phosphorylation, JNK phosphorylation, or both MKK phosphorylation and JNK phosphorylation.

27. The method of claim 12, wherein detecting a change in JNK/MKK protein complex formation or activity in the presence of the test agent comprises detecting a reduced level of c-Jun polypeptide phosphorylation.

28. The method of claim 12, wherein the test agent is a peptide, a peptidomimetic, a polynucleotide, or a small organic molecule.

29. The method of claim 12, wherein the test agent comprises one of a library of test agents.

30. The method of claim 12, which is performed in a high throughput format.

31. The method of claim 12, which is performed in vitro.

32. The method of claim 12, which is performed in a cell, wherein the JNK and the MKK, and, when present, the scaffold protein, are expressed in the cell, said method comprising contacting the cell with the test agent.

33. The method of claim 32, wherein the cell is a mammalian cell.

34. The method of claim 32, wherein the cell is a synoviocyte.

35. The method of claim 32, wherein the JNK, the MKK, or both the JNK and MKK are endogenous to the cell.

36. The method of claim 35, wherein the scaffold protein, when present, is endogenous to the cell.

37. The method of claim 32, wherein a MAP3K also is expressed in the cell.

38. The method of claim 32, wherein the cell is in culture.

39. The method of claim 32, wherein detecting a change in JNK protein complex formation or activity in the presence of the test agent comprises detecting a change in translocation of the JNK/MKK protein complex in the cell.

40. An agent that modulates JNK mediated signal transduction, said agent obtained by the method of claim 12.

41. A method of ameliorating an arthritis associated with c-Jun N-terminal kinase (JNK) signalsome mediated signal transduction in cells of a subject, the method comprising contacting the cells of the subject with an agent that reduces or inhibits JNK/MAP kinase kinase (MKK) protein complex formation or activity, thereby reducing or inhibiting JNK signalsome mediated signal transduction in the cells and ameliorating the arthritis.

42. The method of claim 41, wherein ameliorating the arthritis comprises reducing or preventing an inflammatory reaction at a site of the arthritis.

43. The method of claim 41, wherein ameliorating the arthritis comprises reducing or preventing bone destruction, cartilage destruction, or bone destruction and cartilage destruction at a site of the arthritis.

44. The method of claim 41, wherein the arthritis is rheumatoid arthritis or osteoarthritis.

45. The method of claim 41, wherein contacting the cells of the subject with the agent comprises administering the agent to the subject.

46. The method of claim 45, wherein the agent is administered to a site of the arthritis in the subject.

47. The method of claim 41, wherein contacting the cells of the subject with the agent is performed ex vivo, the method further comprising administering the cells contacted with the agent to the subject.

48. The method of claim 47, wherein the cells contacted with the agent are administered to a site of the arthritis in the subject.

49. An isolated JNK signalsome protein complex, comprising a c-Jun N-terminal kinase (JNK) polypeptide, a mitogen associated protein (MAP) kinase kinase 4 (MKK4) polypeptide, and an MKK7 polypeptide.

50. The isolated JNK signalsome of claim 49, further comprising a scaffold protein.

51. The isolated JNK signalsome of claim 50, wherein the scaffold protein comprises a JNK interacting protein (JIP).

52. An antibody that specifically binds the JNK signalsome of claim 49, wherein the antibody does not substantially bind any of an isolated JNK polypeptide, an isolated MKK4 polypeptide, or an isolated MKK7 polypeptide.

53. An antibody that specifically binds the JNK signalsome of claim 51, wherein the antibody does not substantially bind any of an isolated JNK polypeptide, an isolated MKK4 polypeptide, an isolated MKK7 polypeptide, or an isolated JIP polypeptide.
